(19) United States
(12) Patent Application Publication
(10) Pub. No.: US 2026/0279589 A1
(43) Pub. Date: Sep. 17, 2026

(54) PROTEIN MARKERS FOR MILD COGNITIVE IMPAIRMENT AND ALZHEIMER'S DISEASE

(71) Applicants: The Hong Kong University of Science and Technology, Hong Kong (CN); Hong Kong Center for Neurodegenerative Diseases Limited, Hong Kong SAR (CN)

(72) Inventors: Nancy Yuk-Yu IP, Kowloon, Hong Kong SAR (CN); Yuanbing JIANG, Kowloon, Hong Kong SAR (CN); Hyebin UHM, Kowloon, Hong Kong SAR (CN); Kit Yu FU, Kowloon, Hong Kong SAR (CN); Chui Fun Fanny IP, Kowloon, Hong Kong SAR (CN)

(21) Appl. No.: 19/473,799

(22) PCT Filed: Apr. 12, 2024
(86) PCT No.: PCT/CN2024/087409

§ 371 Date: Oct. 8, 2025

Related U.S. Application Data
(60) Provisional application No. 63/495,864, filed on Apr. 13, 2023.

(51) Int. Cl. G16H 50/30; G01N 33/68; G16B 25/10

(52) U.S. Cl. CPC G16H 50/30; G01N 33/6896; G16B 25/10; G01N 2800/2821; G01N 2800/50

(57) ABSTRACT

The present invention relates to protein markers relevant to mild cognitive impairment (MCI) and Alzheimer's disease (AD), especially those detectable in blood samples. Thus, methods and compositions are provided for risk assessment and early diagnosis of MCI and AD based on the analysis of these protein markers. Further provided are methods and compositions useful for evaluating the efficacy of a therapy for MCI or AD.

### RELATED APPLICATIONS

This application claims priority to U.S. Provisional Patent Application No. 63/495,864, filed Apr. 13, 2023, the contents of which are hereby incorporated by reference in the entirety for all purposes.

### BACKGROUND

Neurodegenerative diseases are devastating conditions of the brain that affect a large subset of the population. Many are highly debilitating, currently incurable, and often result in progressive deterioration of brain structure and cognitive function. For instance, Alzheimer's disease (AD) constitutes 60-80% of dementia cases and represents a leading cause of death in the elderly. Characterized by progressive cognitive decline, AD is an age-related, progressive neurodegenerative disorder that is currently affecting 46.8 million people worldwide, ~10% of people aged 65 or above, with nearly 10 million new cases every year. The pathological hallmarks of this chronic disease include the accumulation of amyloid-beta plaques and neurofibrillary tangles in the brain, together with synaptic dysfunction and neuronal loss, which trigger inflammatory responses in the brain. The most common AD symptoms include memory problems, difficulty communicating, impaired reasoning and judgement, and reduced locomotor abilities. Like many neurodegenerative diseases and neuroinflammatory disorders, current challenges in AD diagnosis attribute to limited understanding of the disease pathophysiology. Meanwhile, currently available treatments are ineffective and can only provide transient effects on symptom alleviation, while patients still suffer severely from the diseases.

Characterized as a transitional state between normal cognition and dementia such as AD, mild cognitive impairment (MCI) accounts for about 10-20% in people over 65 years of age. Individuals with MCI are cognitively impaired but not demented. While at a greater risk of developing AD or other dementia compared to those with normal cognition, they have a cumulative probability of conversion to AD at 33-50%. Whilst MCI is considered a symptomatic pre-dementia stage, its reversion to normal cognition is deemed possible. Hence, to treat MCI and prevent or delay the conversion of MCI to AD, the recognition of MCI is crucial. Specifically, the early diagnosis and timely intervention of AD and pre-AD MCI will advance the prevention and treatment of AD, which is expected to effectively delay the progression of AD and reduce the medical, socioeconomic and psychological burden on families and the society as a whole. The present invention fulfils these and related needs.

### SUMMARY OF THE INVENTION

The invention relates to the discovery of novel protein markers associated with the mild cognitive impairment (MCI) and Alzheimer's disease (AD). The invention thus provides methods and compositions useful for early diagnosis of MCI and AD in a subject. Also provided as methods for evaluating therapeutic efficacy of an agent for MCI and AD treatment.

As such, in a first aspect, the present invention provides a method for assessing risk of developing mild cognitive impairment (MCI) or Alzheimer's disease (AD) in a subject. The method includes the following steps: (a) comparing at least one protein level or concentration in the subject's plasma or serum or whole blood sample with a standard control level of the same protein, the at least one protein is selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3, and the standard control level of the same protein is found in a plasma or serum or whole blood, respectively, of an average healthy subject not suffering from or at risk for MCI or AD; (b) detecting a lower protein level or concentration of AOC3, CA5A, CES1, FCN2, GP1BA, KYNU, or PSME1, or a higher protein level of CCL27, CD27, CD33, CTRC, DCBLD2, IGFBP-2, KIRREL2, LGALS7, NCS1, NEFL, or TNNI3 in the subject's plasma or serum or whole blood sample than the standard control level of the same protein; and (c) determining the subject having an increased risk of developing MCI or AD. In some embodiments, the method further includes, prior to step (a), a step of measuring the at least one protein level or concentration in the subject's plasma or serum or whole blood sample. In some embodiments, prior to the measuring step, the method further includes obtaining the plasma or serum or whole blood sample from the subject. In some embodiments, the step of measuring protein level or concentration involves the use of an antibody-based detection method, an aptamer-based detection method, or a mass spectrometry method. In some embodiments, when the subject is determined in step (c) as having risk of developing MCI or AD, the subject is then provided increased follow-up monitoring (e.g., monitoring tests at an increased frequency compared to the routine monitoring prescribed by a healthcare professional to a no-risk or low-risk person of similar age and medical background). In some embodiments, when the subject is determined in step (c) as having an increased risk of developing MCI or AD, the subject is then administrated with a therapeutic agent for preventing or treating MCI or AD.

While any one of the 18 proteins identified in Table 1 is suitable for use in this method, in some cases multiple proteins (any two, three, four, five, six, seven, eight, nine, ten, eleven, twelve, thirteen, fourteen, fifteen, sixteen, seventeen or more of the 18 proteins) are concurrently examined in this method to achieve better assessment of the risk of developing MCI or AD in a subject. In some embodiments, the concurrent use of multiple proteins (any two, three, four, five, six, seven, eight, nine, ten, eleven, twelve, thirteen, fourteen, fifteen, sixteen, seventeen or more of the 18 proteins) in this method can improve accuracy, sensitivity, and specificity in determining the risk of developing MCI or AD. In some cases, the concurrent use of multiple proteins (any two, three, four, five, six, seven, eight, nine, ten, eleven, twelve, thirteen, fourteen, fifteen, sixteen, seventeen or more of the 18 proteins) in this method allows assessment of multiple biological pathways/systems, thus providing a more comprehensive evaluation on the subject's disease status.

In some embodiments, a prediction model is used to integrate the level of any two or more proteins from the 18 proteins to predict MCI risk and AD risk. In some embodiments, the prediction model using multiple protein markers in predicting MCI risks and AD risks achieves a better diagnostic performance than a method using any single protein from the 18 proteins. In some embodiments, the prediction model uses any two proteins from the 18 proteins. In some embodiments, the prediction model uses any three proteins from the 18 proteins. In some embodiments, the prediction model uses any four or more proteins from the 18 proteins. In some instances, at least two proteins from the group of 18 proteins are selected and measured for risk assessment in accordance with the claimed method. In some instances, at least three or more proteins from the group of 18 proteins are selected and then assessed in accordance with the claimed method.

In a second aspect, the present invention provides a kit for assessing risk of developing mild cognitive impairment (MCI) or Alzheimer's disease (AD) in a subject or for assessing therapeutic efficacy of a treatment regimen for MCI or AD in a subject. The kit includes at least one reagent capable of determining the subject's plasma or serum or whole blood level or concentration of any one, two, three, four, five, six, seven, eight, nine, ten, eleven, twelve, thirteen, fourteen, fifteen, sixteen, seventeen or more proteins independently selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3. In some embodiments, the kit may further include a standard control for each of the proteins, reflecting the level/concentration of the same protein found in the corresponding plasma or serum or whole blood, respectively, of an average healthy subject not suffering from or at increased risk for MCI or AD. In some embodiments, the kit is used to determine the subject's plasma or serum or whole blood level or concentration of at least any two proteins from the 18 proteins. In some embodiments, the kit can determine the subject's plasma or serum or whole blood level or concentration of at least any three, four, five, six, seven, eight, nine, ten, eleven, twelve, thirteen, fourteen, fifteen, sixteen, seventeen or more proteins from the 18 proteins.

In a third aspect, the present invention provides a detection chip for assessing risk of developing mild cognitive impairment (MCI) or Alzheimer's disease (AD) in a subject, or for assessing therapeutic efficacy of a treatment regimen for MCI or AD in a subject. The chip comprises a solid substrate and reagent(s) capable of determining the subject's plasma or serum or whole blood level of any one, two, three, four, five, six, seven, eight, nine, ten, eleven, twelve, thirteen, fourteen, fifteen, sixteen, seventeen or more proteins independently selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3, with each reagent is immobilized at an addressable location on the substrate. In some embodiments, the chip is used to determine the subject's plasma or serum or whole blood level or concentration of at least any two proteins from the 18 proteins. In some embodiments, the chip can determine the subject's plasma or serum or whole blood level or concentration of at least any three, four, five, six, seven, eight, nine, ten, eleven, twelve, thirteen, fourteen, fifteen, sixteen, seventeen or more proteins from the 18 proteins.

In a fourth aspect, the present invention provides a method for quantifying risk of developing mild cognitive impairment (MCI) or Alzheimer's disease (AD) in a subject. The method includes these steps: (a) calculating an individual risk score by inputting a set of values into the formula:

$$Individual risk score = \frac{1}{1 + {e}^{- ( \sum {β}_{i} Candidate {protein}_{i} + ε )}} ,$$

and
(b) determining the subject who has a score lower than an optimal cutoff as having no to low risk or no increased risk of developing MCI or AD, and determining the subject who has a score higher than the optimal cutoff as having a high or an increased risk of developing MCI or AD. In this method, the set of values comprises the plasma or serum or whole blood level of candidate proteins of the 18 proteins set forth in Table 2. In this method, the optimal cutoff for defining low or high risk of developing MCI or AD is determined as the value with the maximum Youden index using the optimal.cutpoints( ) function from the R OptimalCutpoints package, βi is a weighted coefficient of a candidate protein, and ε is the intercept.

In some embodiments, the set of values consists of the plasma or serum or whole blood level of each of the 18 proteins, and the weighted coefficients range (βi) and intercept range (ε) are set forth in Table 2, and a subject with a risk score higher than 0.356 is deemed to have an increased risk of developing MCI or AD. Otherwise the subject is deemed to have low risk or no increased risk for MCI or AD.

In some embodiments, the set of values consists of the plasma or serum or whole blood level of CCL27 and IGFBP-2, and the weighted coefficients range (βi) and intercept range (ε) are set forth in Table 3, and a subject with a risk score higher than 0.656 is deemed to have an increased risk of developing MCI or AD. Otherwise the subject is deemed to have low risk or no increased risk for MCI or AD.

In some embodiments, the set of values consists of the plasma or serum or whole blood level of AOC3, CD27, and NCS1, and the weighted coefficients range (βi) and intercept range (ε) are set forth in Table 4, and a subject with a risk score higher than 0.266 is deemed to have an increased risk of developing MCI or AD. Otherwise the subject is deemed to have low risk or no increased risk for MCI or AD.

In some embodiments, the set of values consists of the plasma or serum or whole blood level of AOC3 and CD27, and the weighted coefficients range (βi) and intercept range (ε) are set forth in Table 5, and a subject with a risk score higher than 0.620 is deemed to have an increased risk of developing MCI or AD. Otherwise the subject is deemed to have low risk or no increased risk for MCI or AD.

In some embodiments, the set of values consists of the plasma or serum or whole blood level of AOC3 and NCS1, and the weighted coefficients range (βi) and intercept range (ε) are set forth in Table 6, and a subject with a risk score higher than 0.833 is deemed to have an increased risk of developing MCI or AD. Otherwise the subject is deemed to have low risk or no increased risk for MCI or AD.

In some embodiments, the set of values consists of the plasma or serum or whole blood level of CD27 and NCS1, and the weighted coefficients range (βi) and intercept range (c) are set forth in Table 7, and a subject with a risk score higher than 0.509 is deemed to have an increased risk of developing MCI or AD. Otherwise the subject is deemed to have low risk or no increased risk for MCI or AD.

In some embodiments, the set of values consists of the plasma or serum or whole blood level of CTRC, KYNU, and TNNI3, and the weighted coefficients range (βi) and intercept range (ε) are set forth in Table 8, and a subject with a risk score higher than 0.489 is deemed to have an increased risk of developing MCI or AD. Otherwise the subject is deemed to have low risk or no increased risk for MCI or AD.

In some embodiments, the set of values consists of the plasma or serum or whole blood level of CTRC and KYNU, and the weighted coefficients range (βi) and intercept range (ε) are set forth in Table 9, and the subject with risk scores higher than 0.589 is deemed to have an increased risk of developing MCI or AD. Otherwise the subject is deemed to have low risk or no increased risk for MCI or AD.

In some embodiments, the set of values consists of the plasma or serum or whole blood level of CTRC and TNNI3, and the weighted coefficients range (βi) and intercept range (ε) are set forth in Table 10, and the subject with risk scores higher than 0.515 is deemed to have an increased risk of developing MCI or AD. Otherwise the subject is deemed to have low risk or no increased risk for MCI or AD.

In some embodiments, the set of values consists of the plasma or serum or whole blood level of KYNU and TNNI3, and the weighted coefficients range (βi) and intercept range (ε) are set forth in Table 11, and the subject with risk scores higher than 0.799 is deemed to have an increased risk of developing MCI or AD. Otherwise the subject is deemed to have low risk or no increased risk for MCI or AD.

In some embodiments, the method further includes, prior to step (a), a step of measuring the plasma or serum or whole blood level of the proteins. In some embodiments, the method in additional includes, prior to the measuring step, another step of obtaining a plasma or serum or whole blood sample from the subject. In some embodiments, when the subject is determined in step (b) as having an increased risk of developing MCI or AD, the subject is then given increased follow-up monitoring (e.g., monitoring tests at an increased frequency compared to the routine monitoring prescribed by a healthcare professional to a no-risk or low-risk person of similar age and medical background) and/or treatment for MCI or AD as described in this disclosure. When the subject is determined as not having any increased risk of developing MCI or AD, the subject is then given the routine monitoring generally prescribed by a physician to a no-risk or low-risk person of developing MCI or AD.

In a fifth aspect, the present invention provides a method for assessing efficacy of a therapeutic agent for treating mild cognitive impairment (MCI) or Alzheimer's disease (AD) in a subject. The method includes these steps: (a) comparing the subject's plasma or serum or whole blood levels of any one protein selected from the proteins named in Table 1 before and after administration of the therapeutic agent to the subject; (b) detecting a decrease in the subject's plasma or serum or whole blood level of CCL27, CD27, CD33, CTRC, DCBLD2, IGFBP-2, KIRREL2, LGALS7, NCS1, NEFL, or TNNI3, or an increase in the subject' plasma or serum or whole blood level of AOC3, CA5A, CES1, FCN2, GP1BA, KYNU, or PSME1 after administration of the therapeutic agent; and (c) determining the therapeutic agent as effective for treating MCI or AD. In some embodiments, the method further includes, prior to step (a), a step of measuring the plasma or serum or whole blood level of the protein or proteins before and after administration. In some embodiments, the method may also include, prior to the measuring step, obtaining a plasma or serum or whole blood sample from the subject before and after administration.

In some embodiments, when the therapeutic agent is deemed in step (c) as effective for treating MCI or AD, the subject will continue the treatment by receiving administration of the therapeutic agent; when the therapeutic agent is deemed in step (c) as not effective for treating MCI or AD, the subject will discontinue the treatment of administration of the therapeutic agent; rather, the subject will initiate another, different treatment by receiving administration of a different therapeutic agent. In some embodiments, the subject is a Chinese descendant.

### BRIEF DESCRIPTION OF THE DRAWINGS

FIG. 1. Prediction of MCI risks and AD risks based on the model utilizing 18 blood proteins. (a) Boxplot showing the individual's risk scores assigned by the 18-protein model (i.e., a model integrating AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3; listed in Table 2) in the HK Chinese cohort, stratified by diagnoses (n=9 CN, 14 MCI and 16 AD, respectively). The dashed line (risk score=0.356) represents the cut-off of having risks of developing MCI and AD (b, c) Receiver operating characteristic (ROC) curve of the 18-protein model (full line) and single protein (dashed line) in differentiating patients with MCI from CN (b) and patients with AD from CN (c) in the HK Chinese cohort. The numbers in the brackets indicate the area under ROC curve of the corresponding models. * P<0.05, ** P<0.01, *** P<0.001.

FIG. 2. Prediction of MCI risks and AD risks based on the model utilizing 2 blood proteins from the 18 blood proteins. (a) Boxplot showing the individual's risk scores assigned by the 2-protein model (i.e., a model integrating CCL27 and IGFBP-2; listed in Table 3) in the HK Chinese cohort, stratified by diagnoses (n=9 CN, 14 MCI and 16 AD, respectively). The dashed line (risk score=0.656) represents the cut-off of having risks of developing MCI and AD. (b, c) Receiver operating characteristic (ROC) curve of the 2-protein model (full line) and single protein (dashed line) in differentiating patients with MCI from CN (b) and patients with AD from CN (c) in the HK Chinese cohort. The numbers in the brackets indicate the area under ROC curve of the corresponding models. * P<0.05, ** P<0.01, *** P<0.001.

FIG. 3. Prediction of MCI risks and AD risks based on the model utilizing 3 blood proteins. (a) Boxplot showing the individual's risk scores assigned by the 3-protein model (i.e., a model integrating AOC3, CD27, and NCS1; listed in Table 4) in the HK Chinese cohort, stratified by diagnoses (n=9 CN, 14 MCI and 16 AD, respectively). The dashed line (risk score=0.266) represents the cut-off of having risks of developing MCI and AD. (b, c) Receiver operating characteristic (ROC) curve of the 3-protein model (full line) and single protein (dashed line) in differentiating patients with MCI from CN (b) and patients with AD from CN (c) in the HK Chinese cohort. The numbers in the brackets indicate the area under ROC curve of the corresponding models. * P<0.05, ** P<0.01, *** P<0.001.

FIG. 4. Prediction of MCI risks and AD risks based on the models utilizing 2 blood proteins from the 3 blood proteins. (a) Boxplot showing the individual's risk scores assigned by the 2-protein model (i.e., a model integrating AOC3 and CD27; listed in Table 5) in the HK Chinese cohort, stratified by diagnoses (n=9 CN, 14 MCI and 16 AD, respectively). The dashed line (risk score=0.620) represents the cut-off of having risks of developing MCI and AD. (b, c) Receiver operating characteristic (ROC) curve of the 2-protein model (full line) and single protein (dashed line) in differentiating patients with MCI from CN (b) and patients with AD from CN (c) in the HK Chinese cohort. The numbers in the brackets indicate the area under ROC curve of the corresponding models. (d) Boxplot showing the individual's risk scores assigned by the 2-protein model (i.e., a model integrating AOC3 and NCS1; listed in Table 6) in the HK Chinese cohort, stratified by diagnoses (n=9 CN, 14 MCI and 16 AD, respectively). The dashed line (risk score=0.833) represents the cut-off of having risks of developing MCI and AD. (e, f) Receiver operating characteristic (ROC) curve of the 2-protein model (full line) and single protein (dashed line) in differentiating patients with MCI from CN (e) and patients with AD from CN (f) in the HK Chinese cohort. The numbers in the brackets indicate the area under ROC curve of the corresponding models. (g) Boxplot showing the individual's risk scores assigned by the 2-protein model (i.e., a model integrating CD27 and NCS1; listed in Table 7) in the HK Chinese cohort, stratified by diagnoses (n=9 CN, 14 MCI and 16 AD, respectively). The dashed line (risk score=0.509) represents the cut-off of having risks of developing MCI and AD. (h, i) Receiver operating characteristic (ROC) curve of the 2-protein model (full line) and single protein (dashed line) in differentiating patients with MCI from CN (h) and patients with AD from CN (i) in the HK Chinese cohort. The numbers in the brackets indicate the area under ROC curve of the corresponding models. * P<0.05, ** P<0.01, *** P<0.001.

FIG. 5. Prediction of MCI risks and AD risks based on the model utilizing 3 blood proteins. (a) Boxplot showing the individual's risk scores assigned by the 3-protein model (i.e., a model integrating CTRC, KYNU, and TNNI3; listed in Table 8) in the HK Chinese cohort, stratified by diagnoses (n=9 CN, 14 MCI and 16 AD, respectively). The dashed line (risk score=0.489) represents the cut-off of having risks of developing MCI and AD. (b, c) Receiver operating characteristic (ROC) curve of the 3-protein model (full line) and single protein (dashed line) in differentiating patients with MCI from CN (b) and patients with AD from CN (c) in the HK Chinese cohort. The numbers in the brackets indicate the area under ROC curve of the corresponding models. * P<0.05, ** P<0.01, *** P<0.001.

FIG. 6. Prediction of MCI risks and AD risks based on the models utilizing 2 blood proteins from the 3 blood proteins. (a) Boxplot showing the individual's risk scores assigned by the 2-protein model (i.e., a model integrating CTRC and KYNU; listed in Table 9) in the HK Chinese cohort, stratified by diagnoses (n=9 CN, 14 MCI and 16 AD, respectively). The dashed line (risk score=0.589) represents the cut-off of having risks of developing MCI and AD. (b, c) Receiver operating characteristic (ROC) curve of the 2-protein model (full line) and single protein (dashed line) in differentiating patients with MCI from CN (b) and patients with AD from CN (c) in the HK Chinese cohort. The numbers in the brackets indicate the area under ROC curve of the corresponding models. (d) Boxplot showing the individual's risk scores assigned by the 2-protein model (i.e., a model integrating CTRC and TNNI3; listed in Table 10) in the HK Chinese cohort, stratified by diagnoses (n=9 CN, 14 MCI and 16 AD, respectively). The dashed line (risk score=0.515) represents the cut-off of having risks of developing MCI and AD. (e, f) Receiver operating characteristic (ROC) curve of the 2-protein model (full line) and single protein (dashed line) in differentiating patients with MCI from CN (e) and patients with AD from CN (f) in the HK Chinese cohort. The numbers in the brackets indicate the area under ROC curve of the corresponding models. (g) Boxplot showing the individual's risk scores assigned by the 2-protein model (i.e., a model integrating KYNU and TNNI3; listed in Table 11) in the HK Chinese cohort, stratified by diagnoses (n=9 CN, 14 MCI and 16 AD, respectively). The dashed line (risk score=0.799) represents the cut-off of having risks of developing MCI and AD. (h, i) Receiver operating characteristic (ROC) curve of the 2-protein model (full line) and single protein (dashed line) in differentiating patients with MCI from CN (h) and patients with AD from CN (i) in the HK Chinese cohort. The numbers in the brackets indicate the area under ROC curve of the CORRESPONDING MODELS. * P<0.05, ** P<0.01, *** P<0.001.

### DEFINITIONS

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

In this disclosure the term “biological sample” or “sample” includes sections of tissues such as biopsy and autopsy samples, and frozen sections taken for histologic purposes, or processed forms of any of such samples. Biological samples include blood and blood fractions or products (e.g., whole blood, acellular fraction of blood (serum, plasma), and blood cells), sputum or saliva, lymph and tongue tissue, cultured cells, e.g., primary cultures, explants, and transformed cells, stool, urine, stomach biopsy tissue etc. A biological sample is typically obtained from a eukaryotic organism, which may be a mammal, may be a primate and may be a human subject.

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.

The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′₂, a dimer of Fab which itself is a light chain joined to V_{H}-C_{H}1 by a disulfide bond. The F(ab)′₂ can be reduced under mild conditions to break the disulfide linkage in the hinge region thereby converting the (Fab′)₂ dimer into an Fab′ monomer. The Fab′ monomer is essentially a Fab with part of the hinge region (see, e.g., Fundamental Immunology, Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.

As used in this application, an “increase” or a “decrease” refers to a detectable positive or negative change in quantity from a comparison control, e.g., an established standard control (such as an average level/amount of a particular protein found in samples from healthy subjects who has not been diagnosed with MCI or AD and has no increased risk for MCI or AD). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

The term “amount” as used in this application refers to the quantity of a substance of interest, such as a protein of interest, present in a sample. Such quantity may be expressed in the absolute terms, i.e., the total quantity of the substance in the sample, or in the relative terms, i.e., the concentration of the substance in the sample.

The terms “subject,” “individual,” and “patient” are used interchangeably herein to refer to a mammal, preferably a human, who seeks medical attention due to risk of (e.g., with family history), or having been diagnosed of, MCI or AD. Subjects also include individuals currently undergoing therapy that seek manipulation of the therapeutic regimen. Subjects or individuals in need of treatment include those that demonstrate symptoms of MCI or AD or are at risk of suffering from MCI or AD or its symptoms. For example, a subject includes an individual with a genetic predisposition or family history for MCI or AD, an individual who has suffered relevant symptoms in the past, an individual who has been exposed to a triggering substance or event, as well as an individual suffering from chronic or acute symptoms of the condition. A subject may be of either gender and at any age of life.

The term “treat” or “treating” as used in this application, describes an act that leads to the elimination, reduction, alleviation, reversal, prevention and/or delay of onset or recurrence of any symptom of a predetermined medical condition. In other words, “treating” a condition encompasses both therapeutic and prophylactic intervention against the condition.

The term “effective amount” as used herein, refers to an amount that produces therapeutic effects for which a substance is administered. The effects include the prevention, correction, or inhibition of progression of the symptoms of a disease/condition and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “standard control” as used herein, refers to a sample comprising an analyte of a predetermined amount to indicate the quantity or concentration of this analyte present in this type of sample (e.g., a predetermined DNA/mRNA or protein) taken from an average healthy subject not suffering from or at risk of developing a predetermined disease or condition (e.g., MCI or AD). When used in the context of describing a value, this term may also be used to simply refer to the quantity or concentration of this analyte present in a “standard control” sample.

The term “average” as used in the context of describing a healthy subject who does not suffer from and is not at risk of developing a relevant disease or disorders (e.g., MCI or AD) refers to certain characteristics, such as the level of a pertinent protein in the person's sample (e.g., serum or plasma or whole blood), that are representative of a randomly selected group of healthy humans who are not suffering from and is not at risk of developing the disease or disorder. This selected group should comprise a sufficient number of human subjects such that the average amount or concentration of the analyte of interest among these individuals reflects, with reasonable accuracy, the corresponding profile in the general population of healthy people. Optionally, the selected group of subjects may be chosen to have a similar background to that of a person whose is tested for indication or risk of the relevant disease or disorder, for example, matching or comparable age, gender, ethnicity, and medical history, etc.

As used herein, the term “Chinese” refers to ethnic Chinese people who and whose ancestors have been residing in the historical territories of China, including the mainland and Hong Kong, for a length of time, e.g., at least the last 3, 4, 5, 6, 7, or 8 generations or the last 100, 150, 200, 250, or 300 years.

### DETAILED DESCRIPTION OF THE INVENTION

### I. Introduction

The present disclosure provides novel protein markers for early diagnosis of or risk assessment for mild cognitive impairment (MCI) and Alzheimer's disease (AD) in a subject. In particular, a panel of 18 protein biomarkers representative of “MCI and/or AD signature” are identified in blood samples that are differentially expressed between heathy subjects and individuals with MCI or AD. The present disclosure provides methods and compositions useful for early diagnosis of MCI or AD in a subject. The present disclosure also provides methods and compositions for evaluating therapeutic treatment of MCI or AD in a subject.

### II. Protein Markers

1. Protein CTRC, also known as chymotrypsin C or caldecrin, is a protease enzyme that is primarily found in the pancreas. It belongs to the family of serine proteases and plays a key role in the regulation of digestive enzymes in the pancreas. Specifically, CTRC helps to activate other pancreatic enzymes such as trypsinogen, which is important for proper digestion of proteins in the small intestine. Mutations in the CTRC gene have been linked to an increased risk for developing chronic pancreatitis, a disease that causes inflammation and damage to the pancreas over time.

2. Protein NCS1, also known as Neuronal Calcium Sensor 1, is a calcium binding protein that is predominantly expressed in the brain and nervous system. NCS1 is involved in a wide range of physiological processes including learning and memory, motor coordination, and sensory processing. Mutations in the NCS1 gene have been associated with certain neurological disorders such as schizophrenia, bipolar disorder, and Parkinson's disease.

3. Protein PSME1, also known as PA28 alpha or REG alpha, is a regulatory protein that is involved in the immune response and cellular stress response. It belongs to the family of proteasome activators and plays a key role in the activation of the 20S proteasome, which is responsible for degrading damaged or misfolded proteins in the cell. PSME1 specifically binds to the 20S proteasome and enhances its proteolytic activity, thereby promoting the clearance of abnormal proteins. PSME1 has also been shown to play a role in antigen processing and presentation, which is important for the recognition and elimination of foreign pathogens by the immune system. Dysregulation of PSME1 expression has been implicated in the development and progression of various diseases, including cancer, autoimmune disorders, and neurodegenerative diseases.

4. Protein KYNU, also known as kynureninase, is an enzyme that is involved in the metabolism of tryptophan, an essential amino acid. KYNU catalyzes the conversion of kynurenine to anthranilic acid in the kynurenine pathway, which is the major pathway for tryptophan metabolism in humans. This pathway plays a crucial role in regulating immune function, inflammation, and neurotransmitter synthesis in the brain. Dysregulation of KYNU activity has been implicated in the pathogenesis of various diseases, including autoimmune disorders, neurodegenerative diseases, and cancer. KYNU has also been proposed as a potential therapeutic target for these diseases due to its involvement in modulating immune responses and inflammation.

5. Protein TNNI3, also known as Cardiac Troponin I, is a regulatory protein that is predominantly expressed in cardiac muscle. It is a component of the troponin complex, which is responsible for regulating muscle contraction in response to calcium signaling. TNNI3 specifically binds to actin filaments in the sarcomere and inhibits the interaction between actin and myosin, thereby preventing muscle contraction. It is an important biomarker for diagnosing acute myocardial infarction, commonly known as a heart attack, as its levels in the blood are elevated in response to cardiac muscle damage. Mutations in the TNNI3 gene have been linked to various cardiac disorders, including hypertrophic cardiomyopathy, dilated cardiomyopathy, and restrictive cardiomyopathy.

6. Protein IGFBP2, also known as insulin-like growth factor-binding protein 2, is a binding protein that interacts with insulin-like growth factors (IGFs) to regulate their activity in the body. It is primarily produced in the liver and is found in the circulation. IGFBP2 modulates the bioavailability of IGFs, which are important regulators of cell growth, differentiation, and survival. IGFBP2 has been implicated in a wide range of physiological processes, including embryonic development, tissue repair, and metabolism. Dysregulation of IGFBP2 expression has been associated with various diseases, including cancer, metabolic disorders, and neurodegenerative diseases. IGFBP2 is being investigated as a potential biomarker for the diagnosis and prognosis of certain diseases, as well as a therapeutic target for the treatment of cancer and other disorders.

7. Protein DCBLD2, also known as discoidin, CUB and LCCL domain-containing protein 2, is a transmembrane protein that is expressed in a wide range of tissues, including the brain, heart, and lungs. It belongs to the family of adhesion G protein-coupled receptors and plays a role in cell adhesion, migration, and angiogenesis. Dysregulation of DCBLD2 expression has been associated with various diseases, including cancer, cardiovascular disease, and developmental disorders. DCBLD2 has been proposed as a potential therapeutic target for cancer and other diseases due to its involvement in angiogenesis and cell migration.

8. Protein CCL27, also known as cutaneous T-cell-attracting chemokine (CTACK), is a chemokine that is primarily expressed in the skin. It belongs to the family of CC chemokines and plays a role in the recruitment and activation of immune cells, particularly T cells, to the skin. CCL27 is involved in various physiological processes, including inflammatory responses, wound healing, and skin development. Dysregulation of CCL27 expression has been associated with various skin disorders, such as psoriasis, atopic dermatitis, and skin cancer. CCL27 has been proposed as a potential therapeutic target for these diseases due to its involvement in regulating immune responses in the skin.

9. Protein CD33, also known as Siglec-3, is a transmembrane protein that is primarily expressed on the surface of myeloid cells, including monocytes, macrophages, and dendritic cells. It belongs to the family of sialic acid-binding immunoglobulin-like lectins (Siglecs) and plays a role in regulating immune responses. CD33 binds to sialic acid residues on glycoproteins and glycolipids, which modulates cell signaling and adhesion. CD33 is involved in various physiological processes, including phagocytosis, antigen presentation, and cytokine production. Dysregulation of CD33 expression has been associated with various diseases, including Alzheimer's disease, acute myeloid leukemia, and autoimmune disorders. CD33 has been proposed as a potential therapeutic target for these diseases due to its involvement in regulating immune function and cell signaling.

10. Protein NEFL, also known as Neurofilament Light Chain, is a cytoskeletal protein that is primarily expressed in neurons. It is a component of the neurofilament, which is a filamentous protein network that provides structural support for axons and contributes to their electrical properties. NEFL plays a role in axonal transport and is involved in various physiological processes, including neuronal development, plasticity, and regeneration. Dysregulation of NEFL expression has been associated with various neurological disorders, including amyotrophic lateral sclerosis (ALS), Alzheimer's disease, and peripheral neuropathies. NEFL has been proposed as a potential biomarker for these diseases due to its involvement in axonal damage and degeneration.

11. Protein FCN2, also known as ficolin-2, is a soluble pattern recognition receptor that is part of the innate immune system. It belongs to the family of ficolins and plays a role in recognizing and binding to pathogen-associated molecular patterns (PAMPs) on the surface of microorganisms, such as bacteria, viruses, and fungi. FCN2 is primarily produced in the liver and is found in the circulation. FCN2 is involved in various physiological processes, including host defence, inflammation, and tissue repair. Dysregulation of FCN2 expression has been associated with various infectious and inflammatory diseases, including sepsis, pneumonia, and rheumatoid arthritis. FCN2 is being investigated as a potential biomarker and therapeutic target for these diseases.

12. Protein LGALS7, also known as galectin 7, is a soluble lectin that is involved in various cellular processes, including cell adhesion, apoptosis, and immune regulation. It belongs to the family of galectins, which are carbohydrate-binding proteins that interact with glycoproteins and glycolipids on the surface of cells. LGALS7 is expressed in a wide range of tissues, including the skin, gastrointestinal tract, and immune cells. It is involved in various physiological processes, such as wound healing, inflammation, and cancer progression. Dysregulation of LGALS7 expression has been associated with various diseases, including cancer, inflammatory disorders, and neurodegenerative diseases. LGALS7 has been proposed as a potential biomarker and therapeutic target for these diseases due to its involvement in regulating cell signaling and immune responses.

13. Protein GP1BA, also known as glycoprotein Ib Platelet subunit alpha, is a subunit of the glycoprotein Ib-IX-V complex, which is a receptor complex that is primarily expressed on the surface of platelets. It plays a role in platelet adhesion and aggregation, which is important for normal blood clotting and wound healing. GP1BA specifically binds to von Willebrand factor (vWF), a protein that is involved in the initial stages of platelet adhesion and clot formation. Dysregulation of GP1BA expression or activity has been associated with various bleeding disorders, such as Bernard-Soulier syndrome and platelet-type von Willebrand disease. GP1BA is also being investigated as a potential therapeutic target for preventing thrombosis, which is the formation of blood clots that can lead to stroke, heart attack, and other serious conditions.

14. Protein CES1, also known as carboxylesterase 1, is an enzyme that is primarily expressed in the liver and plays a role in drug metabolism and detoxification. It belongs to the family of serine hydrolases and is involved in the hydrolysis of various ester-containing compounds, including drugs, fatty acids, and cholesterol esters. CES1 is also involved in the metabolism of prodrugs, which are inactive compounds that are converted to active drugs once they are metabolized by the body. Dysregulation of CES1 expression has been associated with various diseases, including metabolic disorders, liver disease, and cancer. CES1 is being investigated as a potential therapeutic target for these diseases, as well as a biomarker for drug efficacy and toxicity.

15. Protein AOC3, also known as diamine oxidase (DAO), is an enzyme that is primarily expressed in the small intestine and kidneys. It belongs to the family of copper-containing amine oxidases and is involved in the catabolism of histamine, a biogenic amine that plays a role in various physiological processes, including immune responses and neurotransmission. Dysregulation of AOC3 expression or activity has been associated with various inflammatory and allergic diseases, such as asthma, migraines, and irritable bowel syndrome. AOC3 is being investigated as a potential therapeutic target for these diseases, as well as a biomarker for their diagnosis and prognosis.

16. Protein CD27 is a transmembrane protein that is primarily expressed on the surface of T cells and plays a role in regulating immune responses. It belongs to the family of tumor necrosis factor (TNF) receptors and interacts with its ligand, CD70, to modulate T cell activation, proliferation, and differentiation. CD27 is involved in various physiological processes, including the development and maintenance of immune memory, as well as the regulation of autoimmune responses. Dysregulation of CD27 expression or activity has been associated with various diseases, including cancer, autoimmune disorders, and infectious diseases. CD27 has been proposed as a potential therapeutic target for these diseases, as well as a biomarker for disease diagnosis and prognosis.

17. Protein KIRREL2, also known as kin of IRRE-like protein 2 or Nephrin-like protein 2, is a transmembrane protein that is primarily expressed in the kidneys, brain, and heart. It belongs to the family of immunoglobulin-like domain-containing proteins and plays a role in cell signaling, cell adhesion, and tissue development. KIRREL2 is involved in the formation and maintenance of the glomerular filtration barrier in the kidneys, which is important for proper kidney function. It also plays a role in the development and function of the central nervous system and the heart. Dysregulation of KIRREL2 expression or activity has been linked to various disorders, including kidney disease, neurological disorders, and cardiovascular disease. KIRREL2 is being investigated as a potential therapeutic target for these diseases, as well as a biomarker for their diagnosis and prognosis.

18. Protein CA5A, also known as carbonic anhydrase 5A, is an enzyme that is primarily expressed in the salivary glands, pancreas, and liver. It belongs to the family of carbonic anhydrases, which are zinc-containing enzymes that catalyze the reversible hydration of carbon dioxide to bicarbonate ions and protons. CA5A is involved in various physiological processes, including acid-base balance, fluid secretion, and electrolyte transport. Dysregulation of CA5A expression or activity has been linked to various diseases, including diabetes, obesity, and liver disease. CA5A is being investigated as a potential therapeutic target for these diseases, as well as a biomarker for their diagnosis and prognosis.

While any one of the 18 proteins is suitable for use in the methods and compositions disclosed herein, in some cases multiple proteins (any two, three, four, five, six, seven, eight, nine, ten, eleven, twelve, thirteen, fourteen, fifteen, sixteen, seventee or all of the 18 proteins) are concurrently examined in a method or a kit or a device to achieve better assessment of the risk of developing MCI or AD in a subject. In some embodiments, the concurrent use of multiple proteins (any two, three, four, five, six, seven, eight, nine, ten or more or all of the 18 proteins) in a method or a kit or a device can improve accuracy, sensitivity and specificity in determining the risk of developing MCI or AD. In some cases, the concurrent use of multiple proteins (any two, three, four, five, six, seven, eight, nine, ten or more or all of the 18 proteins) in a method or a kit or a device allows assessment of multiple biological pathways/systems, thus providing a more comprehensive evaluation on the subject's disease status.

In some embodiments, a prediction model is used to integrate the level of any two or more proteins from the 18 proteins to predict MCI risk or AD risk. In some embodiments, the prediction model using multiple protein markers in predicting MCI risks or AD risks achieves a better diagnostic performance than a method using any single protein from the 18 proteins. In some embodiments, the prediction model uses any two proteins from the 18 proteins. In some embodiments, the prediction model uses any three proteins from the 18 proteins. In some embodiments, the prediction model uses any four or more proteins from the 18 proteins. In some instances, at least two proteins from the group of 18 proteins are selected and measured for risk assessment in accordance with the claimed methods. In some instances, at least three or more proteins from the group of 18 proteins are selected and assessed in accordance with the claimed methods.

In some cases, CCL27 and IGFBP-2 are selected and measured for risk assessment in accordance with the claimed methods. In some cases, any two proteins of AOC3, CD27, and NCS1 are selected and measured for risk assessment in accordance with the claimed methods. In some instances, AOC3 and CD27 are selected and measured for risk assessment for MCI or AD. In other instances, CD27 and NCS1 are selected and measured for risk assessment for MCI or AD. In yet other instances, AOC3 and NCS1 are selected and measured for risk assessment for MCI or AD. In some cases, any two proteins of CTRC, KYNU, and TNNI3 are selected and measured for risk assessment in accordance with the claimed methods. In some instances, CTRC and KYNU are selected and measured for risk assessment for MCI or AD. In other instances, KYNU and TNNI3 are selected and measured for risk assessment for MCI or AD. In yet other instances, CTRC and TNNI3 are selected and measured for risk assessment for MCI or AD.

### III. Quantitation of Marker Proteins

### 1. Obtaining Samples

The first step of practicing the present invention is to obtain a blood sample from a subject being tested for assessing the risk of developing or monitoring for severity or progression of MCI or AD. Samples of the same type should be taken from both a control group (healthy individuals not suffering from MCI or AD and without increased risk for MCI or AD) and a test group (subjects being tested for possible MCI or AD, or for increased risk for MCI or AD, for example). Standard procedures routinely employed in hospitals or clinics are typically followed for this purpose.

For the purpose of detecting the presence/quantity of marker proteins or assessing the risk of developing MCI or AD in test subjects, individual patients' blood samples are taken, and the serum or plasma or whole blood level of pertinent marker protein(s) (e.g., one or more proteins selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3 as identified in Table 1) may be measured and then compared to a standard control. If an increased protein level of CCL27, CD27, CD33, CTRC, DCBLD2, IGFBP-2, KIRREL2, LGALS7, NCS1, NEFL, or TNNI3, or a decreased protein level of AOC3, CA5A, CES1, FCN2, GP1BA, KYNU, or PSME1 (depending on the specific β value of the protein maker as shown in the Tables) is observed when compared to the control level, the test subject is deemed to have MCI or AD, or have an elevated risk of developing MCI or AD.

For the purpose of monitoring disease progression or assessing therapeutic effectiveness in MCI or AD patients, individual patient's blood samples may be taken at different time points, such that the level of individual marker protein(s) (e.g., one or more proteins selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3 as identified in Table 1) can be measured to provide information indicating the state of disease. For instance, when a patient's maker protein level shows a general trend of increasing or decreasing over time, the patient is deemed to be improving in the severity of MCI or AD or the therapy the patient has been receiving is deemed effective (depending on the specific β value of the protein maker as shown in the Tables). A lack of substantial change in a patient's marker protein level would indicate a lack of change in the status of MCI or AD and ineffectiveness of the therapy given to the patient.

Moreover, the present inventors have devised novel calculation methods to produce a composite risk score based on multiple marker protein levels (e.g., CCL27, IGFBP-2, AOC3, CD27, NCS1, CTRC, KYNU, TNNI3, or one or more proteins identified in Table 1) to quantify the risk of developing MCI or AD in an individual or to compare the relative risks of developing MCI or AD between two or more individuals.

### 2. Preparing Samples for Protein Detection

Blood samples from a subject are suitable for the present invention and can be obtained by well-known methods and as described in standard medical literature. In certain applications of this invention, serum or plasma may be the preferred sample type. In other cases, whole blood samples may be used.

A blood sample is obtained from a person to be tested or monitored for MCI or AD using a method of the present invention. Collection of blood sample from an individual is performed in accordance with the standard protocol hospitals or clinics generally follow. An appropriate amount of blood is collected and may be stored according to standard procedures prior to further preparation.

The analysis of marker protein(s) found in a patient's sample according to the present invention may be performed using, e.g., serum or plasma or whole blood. The methods for preparing patient samples for protein extraction/quantitative detection are well known among those of skill in the art.

### 3. Determining the Level of Marker Proteins

A protein of any particular identity, such as CCL27, IGFBP-2, AOC3, CD27, NCS1, CTRC, KYNU, TNNI3, or any one identified in Table 1, can be detected using a variety of immunological assays. In some embodiments, a sandwich assay can be performed by capturing the protein from a test sample with an antibody having specific binding affinity for the protein. The protein then can be detected with a labeled antibody having specific binding affinity for it. Such immunological assays can be carried out using microfluidic devices such as microarray protein chips. A protein of interest (e.g., any one of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3 as identified in Table 1) can also be detected by gel electrophoresis (such as 2-dimensional gel electrophoresis) and western blot analysis using specific antibodies. Alternatively, standard immunohistochemical techniques can be used to detect a given protein (e.g., any one of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3 as identified in Table 1), using the appropriate antibodies. Both monoclonal and polyclonal antibodies (including antibody fragment with desired binding specificity) can be used for specific detection of the polypeptide. Such antibodies and their binding fragments with specific binding affinity to a particular protein (e.g., any one of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3 as identified in Table 1) can be generated by known techniques.

Other methods may also be employed for measuring the level of marker protein(s) in practicing the present invention. For instance, a variety of methods have been developed based on the mass spectrometry technology to quantify target proteins even rapidly and accurately in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8 (2): 787-797.

### IV. Establishing a Standard Control

In order to establish a standard control for practicing the method of this invention, a group of healthy persons free of MCI and AD, or with no increased risk for developing MCI or AD, as conventionally defined by having normal cognition (e.g., MoCA ≥26) is first selected. These individuals are within the appropriate parameters, if applicable, for the purpose of screening for and/or monitoring MCI or AD using the methods of the present invention. Optionally, the individuals are of same gender, similar age, or similar ethnic background to the test subjects.

The healthy status of the selected individuals is confirmed by well-established, routinely-employed methods including, but not limited to, general physical examination of the individuals and general review of their medical history.

Furthermore, the selected group of healthy individuals must be of a reasonable size, such that the average amount/concentration of marker protein(s) in the serum or plasma or whole blood sample obtained from the group can be reasonably regarded as representative of the normal or average level among the general population of healthy people without MCI and AD or without having increased risk for MCI or AD. Preferably, the selected group comprises at least 10, 20, 30, or 50 human subjects.

Once an average value for the marker protein(s) is established based on the individual values found in each subject of the selected healthy control group, this average or median or representative value or profile is considered a standard control. A standard deviation is also determined during the same process. In some cases, separate standard controls may be established for separately defined groups having distinct characteristics such as age, gender, or ethnic background.

### V. Assessment

In one aspect, the present disclosure provides a method for assessing a subject's risk of developing mild cognitive impairment (MCI) or Alzheimer's disease (AD). The method includes the following steps: (a) comparing at least one protein level or concentration in the subject's plasma or serum or whole blood sample with a standard control level of the same protein, the at least one protein selected from the 18 proteins listed in Table 1, and the standard control level of the same protein is found in a plasma or serum or whole blood, respectively, of an average healthy subject not suffering from or at risk for MCI or AD; (b) detecting a lower protein level or concentration of AOC3, CA5A, CES1, FCN2, GP1BA, KYNU, or PSME1, or a higher protein level of CCL27, CD27, CD33, CTRC, DCBLD2, IGFBP-2, KIRREL2, LGALS7, NCS1, NEFL, or TNNI3 in the subject's plasma or serum or whole blood sample than the standard control level of the same protein; and (c) determining the subject having an increased risk of developing MCI or AD. In some embodiments, the method further includes, prior to step (a), a step of measuring the at least one protein level or concentration in the subject's plasma or serum or whole blood sample. In some embodiments, prior to the measuring step, the method further includes obtaining the plasma or serum or whole blood sample from the subject. In some embodiments, the step of measuring protein level or concentration involves the use of an antibody-based detection method, an aptamer-based detection method, or a mass spectrometry method.

In some embodiments, when the subject is determined in step (c) as having risk of developing MCI or AD, the subject is then provided increased follow-up monitoring (e.g., monitoring tests at an increased frequency compared to the routine monitoring prescribed by a healthcare professional to a no-risk or low-risk person of similar age and medical background). In some embodiments, when the subject is determined in step (c) as having an increased risk of developing MCI or AD, the subject is then administrated with a therapeutic agent for preventing or treating MCI or AD. In some embodiments, when the subject is determined in step (c) as not having any increased risk of developing MCI or AD, the subject is then given the routine monitoring generally prescribed by a physician to a no-risk or low-risk person of developing MCI or AD. Patients of any ethnicity, including the Chinese, are suitable for assessment by the claimed method. In some embodiments, the subject is a Chinese descendant.

In another aspect, the present invention provides a method for quantifying risk of developing mild cognitive impairment (MCI) or Alzheimer's disease (AD) in a subject. The method includes these steps: (a) calculating an individual risk score by inputting a set of values into the formula:

$$Individual risk score = \frac{1}{1 + {e}^{- ( \sum {β}_{i} Candidate {protein}_{i} + ε )}} ,$$

And (b) determining the subject who has a score lower than an optimal cutoff as having low risk of developing MCI or AD, and determining the subject who has a score higher than the optimal cutoff as having an increased risk of developing MCI or AD. In this method, the set of values comprises the plasma or serum or whole blood level of candidate proteins of the 18 proteins set forth in Table 2. In this method, the optimal cutoff for defining low or high risk of developing MCI or AD is determined as the value with the maximum Youden index using the optimal.cutpoints( ) function from the R OptimalCutpoints package, βi is a weighted coefficient of a candidate protein, and & is the intercept.

In some embodiments, the set of values consists of the plasma or serum or whole blood level of each of the 18 proteins as listed in Table 1, and the weighted coefficients range (βi) and intercept range (ε) are set forth in Table 2, and a subject with a risk score higher than 0.356 is deemed to have an increased risk of developing MCI and AD.

In some embodiments, the set of values consists of the plasma or serum or whole blood level of CCL27 and IGFBP-2, and the weighted coefficients range (βi) and intercept range (ε) are set forth in Table 3, and a subject with a risk score higher than 0.656 is deemed to have an increased risk of developing MCI and AD.

In some embodiments, the set of values consists of the plasma or serum or whole blood level of AOC3, CD27, and NCS1, and the weighted coefficients range (βi) and intercept range(s) are set forth in Table 4, and a subject with a risk score higher than 0.266 is deemed to have an increased risk of developing MCI and AD.

In some embodiments, the set of values consists of the plasma or serum or whole blood level of AOC3 and CD27, and the weighted coefficients range (βi) and intercept range (ε) are set forth in Table 5, and a subject with a risk score higher than 0.620 is deemed to have an increased risk of developing MCI and AD.

In some embodiments, the set of values consists of the plasma or serum or whole blood level of AOC3 and NCS1, and the weighted coefficients range (βi) and intercept range(s) are set forth in Table 6, and a subject with a risk score higher than 0.833 is deemed to have an increased risk of developing MCI and AD.

In some embodiments, the set of values consists of the plasma or serum or whole blood level of CD27 and NCS1, and the weighted coefficients range (βi) and intercept range(s) are set forth in Table 7, and a subject with a risk score higher than 0.509 is deemed to have an increased risk of developing MCI and AD.

In some embodiments, the set of values consists of the plasma or serum or whole blood level of CTRC, KYNU, and TNNI3, and the weighted coefficients range (βi) and intercept range(s) are set forth in Table 8, and a subject with a risk score higher than 0.489 is deemed to have an increased risk of developing MCI and AD.

In some embodiments, the set of values consists of the plasma or serum or whole blood level of CTRC and KYNU, and the weighted coefficients range (βi) and intercept range (ε) are set forth in Table 9, and the subject with risk scores higher than 0.589 is deemed to have an increased risk of developing MCI and AD.

In some embodiments, the set of values consists of the plasma or serum or whole blood level of CTRC and TNNI3, and the weighted coefficients range (βi) and intercept range (ε) are set forth in Table 10, and the subject with risk scores higher than 0.515 is deemed to have an increased risk of developing MCI and AD.

In some embodiments, the set of values consists of the plasma or serum or whole blood level of KYNU and TNNI3, and the weighted coefficients range (βi) and intercept range (ε) are set forth in Table 11, and the subject with risk scores higher than 0.799 is deemed to have an increased risk of developing MCI and AD.

In some embodiments, the method further includes, prior to step (a), a step of measuring the plasma or serum or whole blood level of the proteins. In some embodiments, the method in additional includes, prior to the measuring step, another step of obtaining a plasma or serum or whole blood sample from the subject.

In some embodiments, when the subject is determined in step (b) as having an increased risk of developing MCI or AD, the subject is then given increased follow-up monitoring (e.g., monitoring tests at an increased frequency compared to the routine monitoring prescribed by a healthcare professional to a no-risk or low-risk person of similar age and medical background) and treatment as described in this disclosure. When the subject is determined as not having any increased risk of developing MCI or AD, the subject is then given the routine monitoring generally prescribed by a physician to a no-risk or low-risk person of developing MCI or AD. The subject can be of any ethnicity, including the Chinese, suitable for assessment by the claimed method.

In yet another aspect, the present invention provides a method for assessing efficacy of a therapeutic agent for treating mild cognitive impairment (MCI) or Alzheimer's disease (AD) in a subject. The method includes these steps: (a) comparing the subject's plasma or serum or whole blood levels of any one protein selected from the proteins named in Table 1 before and after administration of the therapeutic agent to the subject; (b) detecting a decrease in the subject's plasma or serum or whole blood level of CCL27, CD27, CD33, CTRC, DCBLD2, IGFBP-2, KIRREL2, LGALS7, NCS1, NEFL, or TNNI3, or an increase in the subject' plasma or serum or whole blood level of AOC3, CA5A, CES1, FCN2, GP1BA, KYNU, or PSME1 after administration of the therapeutic agent; and (c) determining the therapeutic agent as effective for treating MCI or AD. In some embodiments, the method further includes, prior to step (a), a step of measuring the plasma or serum or whole blood level of the protein or proteins before and after administration. In some embodiments, the method may also include, prior to the measuring step, obtaining a plasma or serum or whole blood sample from the subject before and after administration.

In some embodiments, when the therapeutic agent is deemed in step (c) as effective for treating MCI or AD, the subject will continue the treatment by administration of the therapeutic agent; when the therapeutic agent is deemed in step (c) as not effective for treating MCI or AD, the subject will discontinue the treatment by administration of the therapeutic agent; rather, the subject will initiate another treatment by administration of a different therapeutic agent. Individuals of any ethnicity, including the Chinese, are suitable for assessment by the claimed method.

### VI. Monitoring and Treatment

In a related aspect, the present invention also provides treatment methods for MCI or AD patients upon detection of MCI or AD or a heightened risk of later developing MCI or AD. In some embodiments, the method comprises, upon determining a subject at early stage of MCI or AD or as having an increased risk for MCI or AD, administering a treatment to said subject, for example, administering an acetylcholinesterase inhibitor (such as donepezil, galantamine, rivastigmine), memantine, a glutamate receptor blocker, citalopram, fluoxetine, paroxeine, sertraline, trazodone, lorazepam, oxazepam, aripiprazole, clozapine, haloperidol, olanzapine, quetiapine, risperidone, ziprasidone, nortriptyline, tricyclic antidepressants, benzodiazepines, temazepam, zolpidem, zaleplon, chloral hydrate, coenzyme Q10, ubiquinone, coral calcium, Ginkgo biloba, huperzine A, omega-3 fatty acids, phosphatidylserine, or any combination thereof.

In some cases, when the diagnostic method steps described above and herein are completed, optionally with additional diagnostic examination performed to provide further confirmatory information (for example, by brain imaging via CT scan or other imaging techniques to show excessive loss of brain volume, or by testing cognitive capability to show an accelerated decline), and a patient has been determined to either already have MCI or AD (e.g., at an early stage) or is at a significantly increased risk of later developing MCI or AD, suitable therapeutic or prophylactic regimens may be ordered by physicians or other medical professionals to treat the patient, to manage/alleviate the ongoing symptoms, or to delay the future onset of the disease. The U.S. Food and Drug Administration (FDA) has approved a number of cholinesterase inhibitors, including donepezil (Aricept™, the only cholinesterase inhibitor approved to treat all stages of AD, including moderate to severe), rivastigmine (Exelon™, approved to treat mild to moderate AD), galantamine (Razadyne™, mild to moderate patients) and memantine (Namenda™). Donepezil is the only cholinesterase inhibitor approved to treat all stages of AD, including moderate to severe. Any one or more of these drugs can be prescribed for treating patients who have been diagnosed with MCI or AD in accordance with the methods of this invention. Another possibility of treatment is administration of trazodone, which is currently approved for use as an antidepressant and has been reported as an effective agent for ameliorating MCI or AD symptoms.

For patients who are deemed at a high or increased risk for developing MCI or AD in a future time but do not yet exhibit any clinical symptoms, continuous monitoring is also appropriate, especially at an increased frequency. For example, the patients may be subject to more frequently scheduled regular testing (e.g., once every six months, once a year, or once every two years) to detect any accelerated change in their cognitive capabilities. Methods suitable for such regular monitoring include General Practitioner Assessment of Cognition (GPCOG), Mini-Cog, Eight-item Informant Interview to Differentiate Aging and Dementia (AD8), and Short Informant Questionnaire on Cognitive Decline in the Elderly (IQCODE). Furthermore, prophylactic treatment with trazodone may also be recommended.

### VII. Kits and Devices

The invention provides compositions and kits for practicing the methods described herein to assess the pertinent marker protein level in a subject's serum/plasma or whole blood, which can be used for various purposes such as detecting or diagnosing the presence of MCI or AD, determining the risk of developing the condition, and monitoring progression of the condition in a patient, including assessing the therapeutic efficacy of a therapy administered for the condition among patients who have received a diagnosis of the disease and have undergone treatment.

Kits for carrying out assays for determining marker protein levels typically include at least one antibody useful for specific binding to the marker protein amino acid sequence. Optionally, this antibody is labeled with a detectable moiety. The antibody can be either a monoclonal antibody or a polyclonal antibody. In some cases, the kits may include at least two different antibodies, one for specific binding to a marker protein (i.e., the primary antibody) and the other for detection of the primary antibody (i.e., the secondary antibody), which is often attached to a detectable moiety.

Typically, the kits also include an appropriate standard control. The standard controls indicate the average value of marker protein(s) in the serum or plasma or whole blood of healthy subjects not suffering from or at increased risk of developing MCI or AD. In some cases, such standard control may be provided in the form of a set value. In addition, the kits of this invention may provide instruction manuals to guide users in analyzing test samples and assessing the presence or risk of MCI or AD, or disease status/progression in a test subject.

In one aspect, the present invention provides a kit for assessing risk of developing mild cognitive impairment (MCI) or Alzheimer's disease (AD) in a subject or for assessing therapeutic efficacy of a treatment regimen for MCI or AD in a subject. The kit includes at least one reagent capable of determining the subject's plasma or serum or whole blood level or concentration of any one, two, three or more proteins independently selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3. In some embodiments, the kit may further include a standard control for each of the proteins, reflecting the level/concentration of the same protein found in the plasma or serum or whole blood, respectively, of an average healthy subject not suffering from or at risk for MCI or AD. In some embodiments, the kit is used to determine the subject's plasma or serum or whole blood level or concentration of at least any two proteins from the 18 proteins. In some embodiments, the kit can determine the subject's plasma or serum or whole blood level or concentration of at least any three, four, five, six, seven, eight, nine, ten, or more proteins from the 18 proteins.

In a further aspect, the present invention can also be embodied in a device or a system comprising one or more such devices, which is capable of carrying out all or some of the method steps described herein. For instance, the device or system performs the following steps upon receiving a serum or plasma or whole blood sample taken from a subject being tested for detecting MCI or AD, assessing the risk of developing MCI or AD, or assessing the disease status/progression: (a) determining in sample the amount or concentration of one or more of the marker protein(s); (b) comparing the marker protein amount/concentration with a standard control value; and (c) providing an output indicating whether MCI or AD is present in the subject or whether the subject is at increased risk of developing MCI or AD, or whether the patient has a higher risk of later developing MCI or AD relative to another patient being tested. In other cases, the device or system of the invention performs the task of steps (b) and (c), after step (a) has been performed and the amount or concentration from (a) has been entered into the device. Preferably, the device or system is partially or fully automated. In some embodiments, the device comprises a detection chip.

As disclosed herein, the present invention provides a detection chip for assessing risk of developing mild cognitive impairment (MCI) or Alzheimer's disease (AD) in a subject, or for assessing therapeutic efficacy of a treatment regimen for MCI or AD in a subject. The chip comprises a solid substrate and reagent(s) capable of determining the subject's plasma or serum or whole blood level of any one, two, three, four, five, six, seven, eight, nine, ten, eleven, twelve, thirteen, fourteen, fifteen, sixteen, seventeen, or all of the eighteen proteins independently selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3, with each reagent is immobilized at an addressable location on the substrate. In some embodiments, the chip is used to determine the subject's plasma or serum or whole blood level or concentration of at least any two proteins from the 18 proteins. In some embodiments, the chip can determine the subject's plasma or serum or whole blood level or concentration of at least any three, four, five, six, seven, eight, nine, ten, eleven, twelve, thirteen, fourteen, fifteen, sixteen, seventeen, or all of the 18 proteins.

### EXAMPLES

The following examples are provided by way of illustration only and not by way of limitation. Those of skill in the art will readily recognize a variety of non-critical parameters that could be changed or modified to yield essentially the same or similar results.

### Introduction

With the increase in global life expectancy, the incidence of MCI and AD is predicted to skyrocket in the coming decades. In particular, the global prevalence of AD is estimated at 75 million in 2030 and 131 million by 2050, while soaring in China where the largest elderly population resides. In fact, the number of AD cases in China doubled from 3.7 million to 9.2 million from 1990-2010, with a projected 22.5 million cases by 2050. Similarly, the Hong Kong population is also aging rapidly, with an estimated 100,000 people currently living with dementia, and ~333 000 or 11% of the population predicted to suffer from dementia in 2039. While increasing with age, the prevalence of MCI ranges from 9.74-27.8% and 2.48-35.5% for the Chinese and European populations, respectively. Despite the devastating impacts, MCI and AD remain largely underdiagnosed in primary care, with only 19% of patients with a confirmed dementia diagnosis as a result of routine medical care, while the proportion of diagnosis or medical consultation sought is estimated to be even lower in Hong Kong. As increasing studies indicate that the disease process of AD begins up to 20 years before the appearance of recognizable symptoms, such underdiagnoses thus attribute primarily to the challenges and limitations associated with the current diagnosis for dementia, which either relies on subjective assessment of apparent expressed symptoms, costly brain imaging or invasive sampling from the cerebral spinal fluid. This together highlights the importance of the time lag between disease onset and diagnosis for any potential intervention, thus the urgent need for early diagnosis, and the significance of novel biomarkers for the detection of MCI, the progression of MCI to AD, and AD.

To address the current lacks in objective diagnostic tools for early detection, the present disclosure provides novel protein markers for early diagnosis of mild cognitive impairment (MCI) and Alzheimer's disease (AD) in a subject. Through large-scale analysis of the blood samples of Hong Kong Chinese participants that comprising individuals with MCI, individuals with AD, and age- and sex-matched healthy and cognitively normal people, a panel of 18 protein biomarkers representative of “MCI and/or AD signature” were identified in blood samples that are differentially expressed between heathy people and individuals with MCI or AD. The present disclosure provides a simple, ultrasensitive, non-invasive and affordable blood-based technology for early diagnosis of MCI and AD, and also for evaluation of therapeutic treatment of MCI or AD in a subject.

The present disclosure provides novel methods and kits related to the use of plasma or serum or whole blood protein markers or their combinations, to assess individual risks of MCI and AD. The invention relates to the discovery of novel blood protein markers associated with MCI and AD. The invention thus provides methods and compositions useful for the risk prediction of MCI and AD as well as for indicating therapeutic efficacy of an agent for treating MCI and AD. As such, in a first aspect, the present invention provides a method for assessing a subject's risk of developing MCI or AD at a later time. The method includes the following steps: (1) comparing the subject's plasma or serum or whole blood level or concentration of any one protein selected from the 18 protein-panel with a standard control level of the same protein found in the plasma or serum or whole blood, respectively, of an average healthy subject not suffering from or at increased risk for MCI or AD; (2) detecting that the subject's plasma or serum or whole blood level of the protein is higher/lower than the standard control level; and (3) determining the subject as having increased risk for MCI or AD. In some embodiments, the method also includes, prior to step (1), a step of measuring the plasma or serum or whole blood level of the protein. In some embodiments, the measuring step is proceeded by a step of obtaining a plasma or serum or whole blood sample from the subject. In some embodiments, when the subject is determined in step (3) as having increased risk for MCI or AD, the subject is then provided increased follow-up monitoring (e.g., monitoring tests at an increased frequency compared to the routine monitoring prescribed by a healthcare professional to a no-risk or low-risk person of similar age and medical background) or treatment as described in this disclosure.

While any of the 18 proteins identified is suitable for use in this method, the concurrent use of multiple biomarkers to determine disease status, commonly termed “composite biomarker panels,” is an effective method to fully exploit the predictive value of protein candidates. Such composite biomarker panels are widely used to predict, for instance, cardiovascular diseases and aging. Compared to using single proteins alone, a biomarker panel integrating multiple proteins can achieve better performance in classifying disease, i.e. improved accuracy, sensitivity and specificity. Moreover, such model allows examination of the status and activities of multiple biological pathways/systems, providing more comprehensive evaluation on the subject's disease status. For this reason, the present inventors also developed a mixed prediction model that can integrate any two or more proteins from the 18 blood proteins, to predict MCI risks and AD risks. The performance of this mixed prediction model in predicting MCI risks and AD risks is better than the method using any single protein from the 18 proteins. Moreover, the present inventors also specifically developed and optimized the mixed prediction models that can integrate two or three proteins (i.e., models that integrate two or three proteins from AOC3, CD27, CTRC, KYNU, NCS1, and TNNI3), to predict MCI risks and AD risks. The performance of these mixed prediction models in predicting MCI risks and AD risks are better than the method using any single proteins from those selected proteins.

### Materials And Methods

Participant recruitment for the Hong Kong Chinese cohort: A total of 39 Hong Kong Chinese individuals aged 60 years or over, comprising of 16 individuals with Alzheimer's disease (AD), 14 individuals with mild cognitive impairment (MCI), and 9 cognitively normal controls (CN), who visited the Neurology Department of the Prince of Wales Hospital of the Chinese University of Hong Kong were recruited. Participants were clinically diagnosed with AD incorporated by the reference “the American Psychiatric Association's Diagnostic and Statistical Manual of Mental Disorders, Arlington (2013, Fifth Edition)”. All participants underwent medical history assessment, clinical assessment, cognitive and functional assessment incorporated by the reference “Nasreddine, Ziad S., et al. The Montreal Cognitive Assessment, MoCA: a brief screening tool for mild cognitive impairment. Journal of the American Geriatrics Society 53.4 (2005): 695-699”, and neuroimaging assessment by amyloid positron emission tomography (PET) imaging using ¹¹C-Pittsburgh Compound B (PiB) and magnetic resonance imaging (MRI). Participants with global cortical-to-cerebellum standardized uptake value ratio ≥1.3 were defined as amyloid PET positive. T1-weighted MRI images were processed by AccuBrain IV1.2 (BrainNow Medical Technology) for brain region segmentation and grey matter volume quantification to analyze the MRI data. Individuals with neurological diseases other than AD or psychiatric diseases were excluded from enrollment. The age, sex, body mass index (BMI), years of education, and medical history of each participant were recorded. CN individuals were defined by having normal cognition (MoCA ≥26) and amyloid PET negative; individuals with MCI and AD were defined by having amyloid PET positive, together with the clinical diagnosis of the cognition. This study was approved by the Prince of Wales Hospital of the Chinese University of Hong Kong and the Hong Kong University of Science and Technology. All participants provided written informed consent for study participation and sample collection.

Plasma preparation from blood samples: Whole blood (3 mL) was collected in K3EDTA tubes (VACUETTE) and centrifuged at 2,000 g for 15 minutes to separate the cell pellet and plasma. The plasma was collected, aliquoted, and stored at −80° C. until use.

Measurement of blood protein levels: Blood abundance of 18 proteins were quantified in prepared plasma samples, via Proximity Extension Assay technology of Olink Proteomics biomarker panels, including Cardiometabolic, Cardiovascular II, Cardiovascular III, Cell Regulation, Immune Response, Neuro Exploratory, Neurology, Oncology II, Oncology III, and Organ Damage.

Association analysis of blood protein level and MCI or AD: The association between normalized protein level and MCI or AD, adjusting for age, sex, and BMI, was analyzed using the following linear regression model (βᵢ, the weighted coefficient for corresponding factors; ε, the intercept of the linear equation):

$$Normalized protein level ∼ {β}_{1} AD + {β}_{2} MCI + {β}_{3} Age + {β}_{4} Sex + {β}_{5} BMI + ε$$

Blood proteins with weighted coefficients for AD (i.e., β₁) greater or less than 0 were considered as being increased or decreased in AD, respectively. Blood proteins with weighted coefficients for MCI (i.e., (β₂) greater or less than 0 were considered as being increased or decreased in MCI, respectively.

### Calculation of Risk Scores:

For each prediction model, the weighted coefficient (βᵢ) of candidate proteins and intercept (ε) were calculated by fitting the blood levels of candidate proteins and AD diagnosis of participants into the following logistic regression model:

$$Diagnosis ( AD = 1 , CN = 0 ) = \frac{1}{1 + {e}^{- ( \sum {β}_{i} Candidate {protein}_{i} + ε )}}$$

Individual risk scores were calculated using the blood levels of candidate proteins and corresponding weighted coefficient (βᵢ) and intercept (ε) using the following linear model:

$$Individual risk score = \frac{1}{1 + {e}^{- ( \sum {β}_{i} Candidate {protein}_{i} + ε )}}$$

The optimal cutoff for defining low or high risk of developing MCI or AD was determined as the value with the maximum Youden index using the optimal.cutpoints( ) function from the R OptimalCutpoints package.

Evaluation of prediction accuracy: The auc( ) function from the R pROC package was used to evaluate the accuracy of each prediction model or single proteins by calculating the areas under the curve (AUCs) of receiver operating characteristic (ROC) curves.

Data visualization: The investigators performing blood protein measurements were blinded to the diagnosis and phenotypes of participants. All statistical plots were generated using Prism v8.0 (GraphPad).

### Example I: a Model Integrating 18 Blood Proteins Predicts MCI Risks and AD Risks

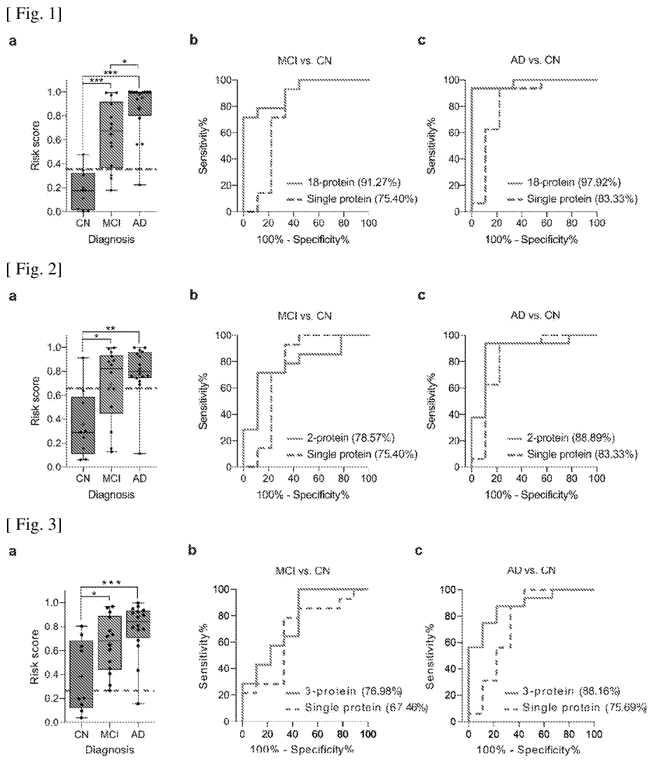

The 18 proteins (i.e., AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3; Table 1) are exhibiting increased (effect size >0) or decreased (effect size <0) blood levels in patients with MCI and/or AD, compared to cognitively normal (CN) people, with the maximum accuracy of 75.40% in differentiating MCI from CN and maximum accuracy of 83.33% in differentiating AD from CN (Table 1). Notably, the present inventors developed a mixed prediction model that integrates the levels of 18 blood proteins and assigns individuals with risk scores (FIG. 1a and Table 2). The resulting scores can well distinguish MCI and CN, with an accuracy of 91.27% (FIG. 1b), and distinguish AD and CN, with an accuracy of 97.92% (FIG. 1c). The performance of this mixed prediction model in predicting MCI risks and AD risks is better than the method using any single protein from the 18 proteins (FIG. 1b, c). Individuals with risk scores lower than 0.356 will have low risk of developing MCI or AD; by comparison, individuals with risk scores larger than 0.356 will have risks of developing MCI and AD (FIG. 1a).

### Example II: a Model Integrating 2 Blood Proteins from the 18 Blood Proteins Predicts MCI Risks and AD Risks

Notably, the present inventors also developed a mixed prediction model that can integrate any two or more proteins from the 18 blood proteins, to predict MCI risks and AD risks. For example, the model integrates the levels of 2 blood proteins (i.e., CCL27 and IGFBP-2) can assign individuals with risks scores (FIG. 2a and Table 3), and the resulting scores can well distinguish MCI and CN, with an accuracy of 78.57% (FIG. 2b), and distinguish AD and CN, with an accuracy of 88.89% (FIG. 2c). The performance of this mixed prediction model in predicting MCI risks and AD risks is better than the method using any single protein from the 18 proteins (FIGS. 2b, 2c). Individuals with risk scores lower than 0.656 will have low risk of developing MCI or AD; by comparison, individuals with risk scores larger than 0.656 will have risks of developing MCI and AD (FIG. 2a).

### Example III: Models Integrating 3 Blood Proteins Predict MCI Risks and AD Risks

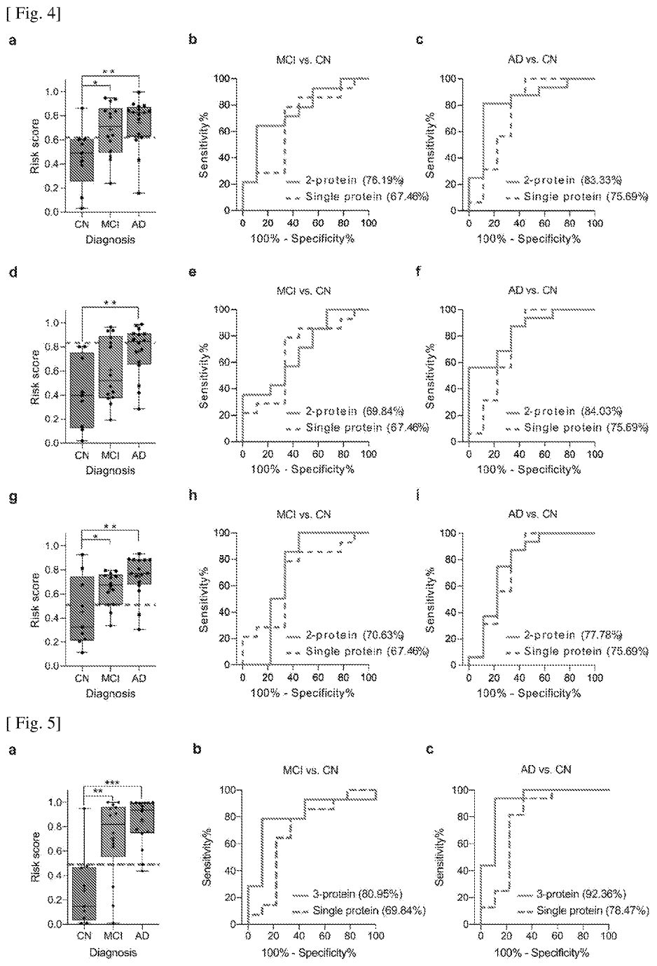

The present inventors also developed a mixed prediction model that can integrate 3 proteins (i.e., AOC3, CD27, and NCS1) to predict MCI risks and AD risks (FIG. 3a and Table 4). The resulting scores can well distinguish MCI and CN, with an accuracy of 76.98% (FIG. 3b), and distinguish AD and CN, with an accuracy of 88.19% (FIG. 3c). The performance of this mixed prediction model in predicting MCI risks and AD risks is better than the method using any single protein from the 3 proteins (FIGS. 3b and 3c). Individuals with risk scores lower than 0.266 will have low risk of developing MCI or AD; by comparison, individuals with risk scores larger than 0.266 will have risks of developing MCI and AD (FIG. 3a). Notably, the present inventors also developed mixed prediction models that can integrate any two of the three proteins to predict MCI risks and AD risks (FIGS. 4a, 4d, 4g and Table 5-7). The resulting scores can well distinguish MCI and CN, with the accuracy from 69.84% to 76.19% (FIGS. 4b, 4e and 4h), and distinguish AD and CN, with the accuracy from 77.78% to 84.03% (FIGS. 4c, 4f and 4i). The performance of these mixed prediction models in predicting MCI risks and AD risks are all better than the method using any single protein from the 3 proteins (FIGS. 4b-4c, 4e-4f, and 4h-4i). Individuals with risk scores lower than 0.620 or 0.833 or 0.509 will have low risk of developing MCI or AD; by comparison, individuals with risk scores larger than 0.620 or 0.833 or 0.509 will have risks of developing MCI and AD (FIGS. 4a, 4d and 4g).

### Example IV: Models Integrating 3 Blood Proteins Predict MCI Risks and AD Risks

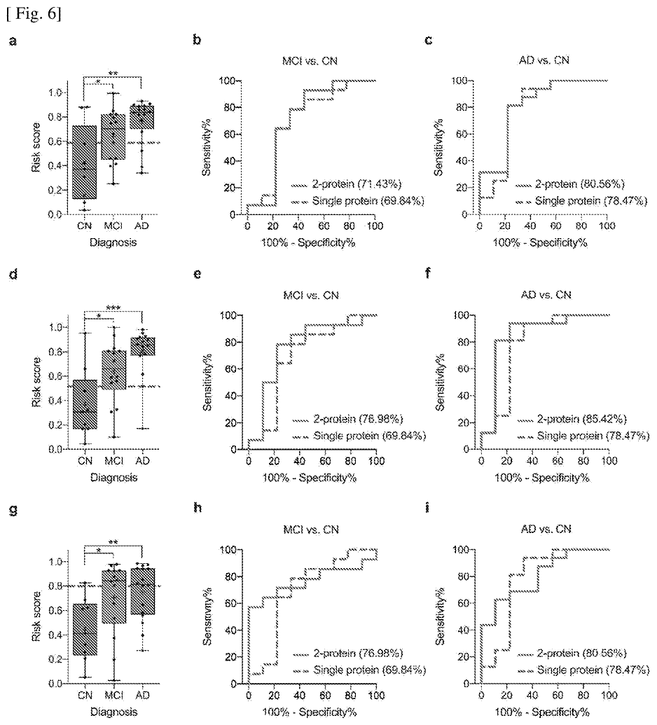

Furthermore, the present inventors developed an additional mixed prediction model that can integrate 3 other proteins (i.e., CTRC, KYNU, and TNNI3) to predict MCI risks and AD risks (FIG. 5a and Table 8). The resulting scores can well distinguish MCI and CN, with an accuracy of 80.95% (FIG. 5b), and distinguish AD and CN, with an accuracy of 92.36% (FIG. 5c). The performance of this mixed prediction model in predicting MCI risks and AD risks is more accurate than models using any single protein from the 3 proteins (FIGS. 5b and 5c). Individuals with risk scores lower than 0.489 will have low risk of developing MCI or AD, while individuals with risk scores larger than 0.489 will have risks of developing MCI and AD (FIG. 5a). Notably, the present inventors also developed mixed prediction models that can integrate any two of the three proteins to predict MCI risks and AD risks (FIGS. 6a, 6d, 6g and Table 9-11). The resulting scores can well distinguish MCI and CN, with the accuracy from 71.43% to 76.98% (FIGS. 6b, 6e and 6h), and distinguish AD and CN, with the accuracy from 80.56% to 85.42% (FIGS. 6c, 6f and 6i). The performance of these mixed prediction models in predicting MCI risks and AD risks are all better than the method using any single protein from the 3 proteins (FIGS. 6b-6c, 6e-6f, and 6h-6i). Individuals with risk scores lower than 0.589 or 0.515 or 0.799 will have low risk of developing MCI or AD; by comparison, individuals with risk scores larger than 0.589 or 0.515 or 0.799 will have risks of developing MCI and AD (FIGS. 6a, 6d and 6g).

All patents, patent applications, and other publications, including GenBank Accession Numbers, Uniprot IDs, and equivalents, cited in this application are incorporated by reference in the entirety for all purposes.

TABLE 1
List of 18 blood proteins that are associated with MCI and
AD. AUC, area under the receiver operating characteristic
curve, which represents the accuracy in classification.

| | Uniprot | Effect size | AUC | Effect size | AUC |
|---|---|---|---|---|---|
| Protein name | ID | (MCI vs. CN) | (MCI vs. CN) | (AD vs. CN) | (AD vs. CN) |
| AOC3 | Q16853 | −0.071 | 67.46 | −0.085 | 70.83 |
| CA5A | P35218 | −0.178 | 61.90 | −0.773 | 77.78 |
| CCL27 | Q9Y4X3 | 0.344 | 75.40 | 0.296 | 70.14 |
| CD27 | P26842 | 0.274 | 65.08 | 0.369 | 68.75 |
| CD33 | P20138 | 0.249 | 63.49 | 0.539 | 72.92 |
| CES1 | P23141 | −0.051 | 50.00 | −0.359 | 79.17 |
| CTRC | Q99895 | 0.426 | 69.84 | 0.593 | 78.47 |
| DCBLD2 | Q96PD2 | 0.058 | 63.49 | 0.166 | 72.22 |
| FCN2 | Q15485 | −0.106 | 53.97 | −0.512 | 74.31 |
| GP1BA | P07359 | −0.028 | 50.79 | −0.089 | 66.67 |
| IGFBP-2 | P18065 | 0.446 | 70.63 | 0.698 | 82.64 |
| KIRREL2 | Q6UWL6 | 0.037 | 59.52 | 0.144 | 83.33 |
| KYNU | Q16719 | −0.355 | 69.05 | −0.451 | 77.08 |
| LGALS7 | P47929 | 0.117 | 60.32 | 0.279 | 67.36 |
| NCS1 | P62166 | 0.179 | 64.29 | 0.366 | 75.69 |
| NEFL | P07196 | −0.050 | 52.38 | 0.264 | 70.83 |
| PSME1 | Q06323 | 0.050 | 56.35 | −0.164 | 68.75 |
| TNNI3 | P19429 | 0.275 | 56.35 | 0.329 | 61.11 |

TABLE 2
Weighted coefficients range (βᵢ) and intercept range
(ε) for the model utilizing 18 blood proteins

| | |
|---|---|
| Intercept range (ε) | 33.66 |

| | Protein name | βᵢ |
|---|---|---|
| Weighted coefficients | AOC3 | −8.294 |
| range (βᵢ) | CA5A | −0.048 |
| | CCL27 | 0.978 |
| | CD27 | 1.189 |
| | CD33 | 0.082 |
| | CES1 | −2.044 |
| | CTRC | 2.841 |
| | DCBLD2 | 0.052 |
| | FCN2 | 0.016 |
| | GP1BA | −1.587 |
| | IGFBP-2 | 1.404 |
| | KIRREL2 | 1.519 |
| | KYNU | −5.344 |
| | LGALS7 | 0.051 |
| | NCS1 | −2.664 |
| | NEFL | 2.798 |
| | PSME1 | −0.160 |
| | TNNI3 | 2.565 |

TABLE 3
Weighted coefficients range (βᵢ) and intercept range (ε) for
the model utilizing 2 blood proteins from the 18 blood proteins

| | |
|---|---|
| Intercept range (ε) | −31.24 |

| | Protein name | βᵢ |
|---|---|---|
| Weighted coefficients | CCL27 | 2.193 |
| range (βᵢ) | IGFBP-2 | 3.131 |

TABLE 4
Weighted coefficients range (βᵢ) and intercept range
(ε) for the model utilizing 3 blood proteins

| | |
|---|---|
| Intercept range (ε) | −2.399 |

| | Protein name | βᵢ |
|---|---|---|
| Weighted coefficients | AOC3 | −8.924 |
| range (βᵢ) | CD27 | −1.613 |
| | NCS1 | 3.138 |

TABLE 5
Weighted coefficients range (βᵢ) and intercept range (ε) for
the model utilizing 2 blood proteins from the 3 blood proteins

| | |
|---|---|
| Intercept range (ε) | 9.348 |

| | Protein name | βᵢ |
|---|---|---|
| Weighted coefficients | AOC3 | −8.179 |
| range (βᵢ) | CD27 | 2.889 |

TABLE 6
Weighted coefficients range (βᵢ) and intercept range (ε) for
the model utilizing 2 blood proteins from the 3 blood proteins

| | |
|---|---|
| Intercept range (ε) | −2.285 |

| | Protein name | βᵢ |
|---|---|---|
| Weighted coefficients | AOC3 | −7.852 |
| range (βᵢ) | NCS1 | 4.170 |

TABLE 7
Weighted coefficients range (βᵢ) and intercept range (ε) for
the model utilizing 2 blood proteins from the 3 blood proteins

| | |
|---|---|
| Intercept range (ε) | −28.95 |

| | Protein name | βᵢ |
|---|---|---|
| Weighted coefficients | CD27 | 1.204 |
| range (βᵢ) | NCS1 | 2.678 |

TABLE 8
Weighted coefficients range (βᵢ) and intercept range
(ε) for the model utilizing 3 blood proteins

| | |
|---|---|
| Intercept range (ε) | −6.964 |

| | Protein name | βᵢ |
|---|---|---|
| Weighted coefficients | CTRC | 3.343 |
| range (βᵢ) | KYNU | −4.516 |
| | TNNI3 | 1.815 |

TABLE 9
Weighted coefficients range (βᵢ) and intercept range (ε) for
the model utilizing 2 blood proteins from the 3 blood proteins

| | |
|---|---|
| Intercept range (ε) | −18.69 |

| | Protein name | βᵢ |
|---|---|---|
| Weighted coefficients | CTRC | 2.460 |
| range (βᵢ) | KYNU | −0.948 |

TABLE 10
Weighted coefficients range (βᵢ) and intercept range (ε) for
the model utilizing 2 blood proteins from the 3 blood proteins

| | |
|---|---|
| Intercept range (ε) | −40.60 |

| | Protein name | βᵢ |
|---|---|---|
| Weighted coefficients | CTRC | 3.490 |
| range (βᵢ) | TNNI3 | 0.642 |

TABLE 11
Weighted coefficients range (βᵢ) and intercept range (ε) for
the model utilizing 2 blood proteins from the 3 blood proteins

| | |
|---|---|
| Intercept range (ε) | 25.49 |

| | Protein name | βᵢ |
|---|---|---|
| Weighted coefficients | KYNU | −3.590 |
| range (βᵢ) | TNNI3 | 1.023 |

What is claimed is:

1. A method for assessing risk of developing mild cognitive impairment (MCI) or Alzheimer's disease (AD) in a subject, comprising the steps of:
(a) comparing at least one protein level in the subject's plasma or serum or whole blood sample with a standard control level of the same protein, wherein the at least one protein is selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3;
(b) detecting a lower protein level of AOC3, CA5A, CES1, FCN2, GP1BA, KYNU, or PSME1, or a higher protein level of CCL27, CD27, CD33, CTRC, DCBLD2, IGFBP-2, KIRREL2, LGALS7, NCS1, NEFL, or TNNI3 in the subject's plasma or serum or whole blood sample than the standard control level of the same protein; and
(c) determining the subject having an increased risk of developing MCI or AD.

2. The method of claim 1, wherein prior to step (a), further comprising measuring the at least one protein level in the subject's plasma or serum or whole blood sample.

3. The method of claim 2, wherein prior to the measuring step, further comprising obtaining the plasma or serum or whole blood sample from the subject.

4. The method of any one of claims 1-3, wherein after step (c), further comprising monitoring the at least one protein level of the subject in an increased frequency.

5. The method of any one of claims 1-3, wherein after step (c), further comprising administrating to the subject a therapeutic agent for preventing or treating MCI or AD.

6. The method of any one of claims 1-5, wherein level of at least two proteins is measured.

7. The method of any one of claims 1-6, wherein level of at least three proteins is measured.

8. A kit for assessing risk of developing mild cognitive impairment (MCI) or Alzheimer's disease (AD) in a subject, comprising a reagent capable of determining level of each of any 1, 2, 3 or more proteins selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3 in the subject's plasma or serum or whole blood.

9. The kit of claim 8, further comprising a standard control for each of the proteins.

10. A detection chip for assessing risk of developing mild cognitive impairment (MCI) or Alzheimer's disease (AD) in a subject, comprising a solid substrate and a reagent capable of determining the subject's plasma or serum or whole blood level of any 1, 2, 3 or more proteins independently selected from the group consisting of AOC3, CA5A, CCL27, CD27, CD33, CES1, CTRC, DCBLD2, FCN2, GP1BA, IGFBP-2, KIRREL2, KYNU, LGALS7, NCS1, NEFL, PSME1, and TNNI3, wherein each reagent is immobilized at an addressable location on the substrate.

11. A method for quantifying risk of developing mild cognitive impairment (MCI) or Alzheimer's disease (AD) in a subject, comprising:
(a) calculating an individual risk score by inputting a set of values into the formula:
$$Individual risk score = \frac{1}{1 + {e}^{- ( \sum {β}_{i} Candidate {protein}_{i} + ε )}} ,$$
and
(b) determining the subject who has a score higher than the optimal cutoff as having risk of developing MCI or AD,
wherein the set of values comprises the plasma or serum or whole blood level of candidate proteins of the 18 proteins set forth in Table 2, wherein the optimal cutoff is determined as the value with the maximum Youden index using an optimal.cutpoints( ) function from a R OptimalCutpoints package, wherein βᵢ is a weighted coefficient of a candidate protein; and wherein & is an intercept.

12. The method of claim 11, wherein the set of values consists of the plasma or serum or whole blood level of each of the 18 proteins, and wherein the weighted coefficients range (βᵢ) and intercept range(s) are set forth in Table 2, and wherein the subject with risk scores higher than 0.356 has risk of developing MCI and AD.

13. The method of claim 11, wherein the set of values consists of the plasma or serum or whole blood level of CCL27 and IGFBP-2, and wherein the weighted coefficients range (βᵢ) and intercept range(s) are set forth in Table 3, and wherein the subject with risk scores higher than 0.656 has risk of developing MCI and AD.

14. The method of claim 11, wherein the set of values consists of the plasma or serum or whole blood level of AOC3, CD27, and NCS1, and wherein the weighted coefficients range (βᵢ) and intercept range(s) are set forth in Table 4, and wherein the subject with risk scores higher than 0.266 has risk of developing MCI and AD.

15. The method of claim 11, wherein the set of values consists of the plasma or serum or whole blood level of two proteins from AOC3, CD27, and NCS1, and wherein the weighted coefficients range (βᵢ) and intercept range (ε) are set forth in Tables 5-7, and wherein the subject with risk scores higher than 0.620 of AOC3 and CD27, or 0.833 of AOC3 and NCS1, or 0.509 of CD27 and NCS1, has risk of developing MCI and AD.

16. The method of claim 11, wherein the set of values consists of the plasma or serum or whole blood level of CTRC, KYNU, and TNNI3, and wherein the weighted coefficients range (βᵢ) and intercept range (ε) are set forth in Table 8, and wherein the subject with risk scores higher than 0.489 has risk of developing MCI and AD.

17. The method of claim 11, wherein the set of values consists of the plasma or serum or whole blood level of two proteins from CTRC, KYNU, and TNNI3, and wherein the weighted coefficients range (βᵢ) and intercept range (ε) are set forth in Tables 9-11, and wherein the subject with risk scores higher than 0.589 of CTRC and KYNU, or 0.515 of CTRC and TNNI3, or 0.799 of KYNU and TNNI3, has risk of developing MCI and AD.

18. The method of any one of claims 11-17, further comprising, prior to step (a), measuring the plasma or serum or whole blood level of the proteins.

19. The method of claim 18, further comprising, prior to the measuring step, obtaining a plasma or serum or whole blood sample from the subject.

20. A method for assessing efficacy of a therapeutic agent for treating mild cognitive impairment (MCI) or Alzheimer's disease (AD) in a subject, comprising:
(a) comparing the subject's plasma or serum or whole blood levels of any one protein selected from Table 1 before and after administration of the therapeutic agent to the subject;
(b) detecting a decrease in the subject's plasma or serum or whole blood level of CCL27, CD27, CD33, CTRC, DCBLD2, IGFBP-2, KIRREL2, LGALS7, NCS1, NEFL, or TNNI3, or an increase in the subject' plasma or serum or whole blood level of AOC3, CA5A, CES1, FCN2, GP1BA, KYNU, or PSME1 after administration of the therapeutic agent; and
(c) determining the therapeutic agent as effective for treating MCI or AD.

21. The method of claim 20, further comprising, prior to step (a), measuring the plasma or serum or whole blood level of the protein before and after administration.

22. The method of claim 21, further comprising, prior to the measuring step, obtaining a plasma or serum or whole blood sample from the subject before and after administration.

23. The method of any one of claims 1-7 and 11-22, wherein the subject is a Chinese descendant.
